(19) United States
(12) Patent Application Publication
(10) Pub. No.: US 2026/0115379 A1
(43) Pub. Date: Apr. 30, 2026

(54) SYSTEMS AND METHODS FOR CALCULATING INSULIN ON BOARD IN INFUSION PUMP SYSTEMS

(71) Applicant: Tandem Diabetes Care, Inc., San Diego, CA (US)

(72) Inventors: Thomas R. ULRICH, Oceanside, CA (US); Nicholas SHERER, Escondido, CA (US); McKenzie Michael Kenneth McManus, Valley Center, CA (US); Jose Ricardo RUEDA, La Jolla, CA (US); Micah Stephens, La Mirada, CA (US); Peter Briggs, El Cajon, CA (US); Peter ZHAO, San Diego, CA (US); Jenna Christine Huseby, Lake Forest, CA (US); William S. Ostrie, II, Encinitas, CA (US)

(21) Appl. No.: 19/003,140

(22) Filed: Dec. 27, 2024

Related U.S. Application Data
(60) Provisional application No. 63/615,541, filed on Dec. 28, 2023.

(51) Int. Cl. A61M 5/142; A61M 5/172

(52) U.S. Cl. CPC A61M 5/14244; A61M 5/1723; A61M 2205/505

(57) ABSTRACT

Disclosed herein are portable infusion pump systems that provide an ability to simultaneously track insulin on board from insulin deliveries from different sources and/or of different types. A user interface can be provided to enable the user to enter insulin delivered from a source other than the portable infusion pump. A simplified calculation for estimating IOB remaining from each insulin delivery enables such calculations to be carried out with the limited memory and processing capacity of the portable infusion pump.

### PRIORITY CLAIM

The present application claims the benefit of U.S. Provisional Patent Application No. 63/615,541 filed Dec. 28, 2023, which is hereby incorporated herein by reference in its entirety.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, which are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as blood glucose meters (BGMs) and continuous glucose monitoring devices (CGMs). A CGM provides a substantially continuous estimated blood glucose level through a transcutaneous sensor that estimates blood analyte levels, such as blood glucose levels, via the patient's interstitial fluid. CGM systems typically consist of a transcutaneously-placed sensor, a transmitter and a monitor.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone. One example of integration of infusion pumps with CGM devices is described in U.S. Patent Publication No. 2014/0276419, which is hereby incorporated by reference herein.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems because the systems serve to mimic biological functions of the pancreas for patients with diabetes. Such systems are also referred to as automated insulin delivery (AID) systems.

An important parameter to be tracked in both closed loop and open loop diabetes therapy is insulin on board, which is the amount of unmetabolized insulin in a user's body that will subsequently lower the user's glucose level. There has been much research into how the body metabolizes insulin, which will vary from user to user because individuals metabolize insulin at slightly different rates. A number of methods for estimating insulin on board have been utilized and insulin pumps as described above will generally track insulin on board for the user. However, the calculations can be resource-intensive and difficult to carry out with the limited capacity of such a pump. In addition, the pump will only be able to account for insulin delivered by the pump and if the user ingests insulin from a source other than the pump, the pump will not be able to account for that unknown insulin.

### SUMMARY

Disclosed herein are portable infusion pump systems that provide an ability to simultaneously track insulin on board from insulin deliveries from different sources and/or of different types. A user interface can be provided to enable the user to enter insulin delivered from a source other than the portable infusion pump. A simplified calculation for estimating IOB remaining from each insulin delivery enables such calculations to be carried out with the limited memory and processing capacity of the portable infusion pump.

In an embodiment, portable infusion pump system can include a pump mechanism configured to facilitate delivery of insulin to a user, a user interface and at least one processor functionally linked to the pump mechanism and the user interface. The at least one processor configured to cause the pump mechanism to deliver the insulin doses to the user and display an indication for user input on the user interface for the portable infusion pump system to receive information pertaining to insulin delivered to the user without use of the pump mechanism. An amount of insulin on board remaining in the user from both the insulin doses delivered with the pump mechanism and the insulin delivered to the user without use of the pump mechanism can be tracked over time. A combination of the remaining insulin on board from the insulin doses delivered with the pump mechanism and the insulin delivered to the user without use of the pump mechanism can be utilized to determine a total insulin on board in making therapy determinations.

In an embodiment, a portable infusion pump system can include a pump mechanism configured to facilitate delivery of insulin to a user and at least one processor functionally linked to the pump mechanism. The at least one processor can be configured to cause the pump mechanism to deliver insulin doses to the user. Information can be received pertaining to insulin delivered to the user without use of the pump mechanism. An amount of insulin on board remaining in the user over time can be tracked from both the insulin doses delivered with the pump mechanism and the insulin delivered to the user without use of the pump mechanism. A combination of the remaining insulin on board from the insulin doses delivered with the pump mechanism and the insulin delivered to the user without use of the pump mechanism can be utilized in making therapy determinations.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the disclosure.

FIG. 2 is a block diagram representing a medical device that can be used with embodiments of the disclosure.

FIGS. 3A-3B depict an embodiment of a pump system according to the disclosure.

FIG. 4 is a schematic representation of a system according to the disclosure.

FIG. 5 depicts an estimate of the unmetabolized insulin burndown of two boluses according to the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

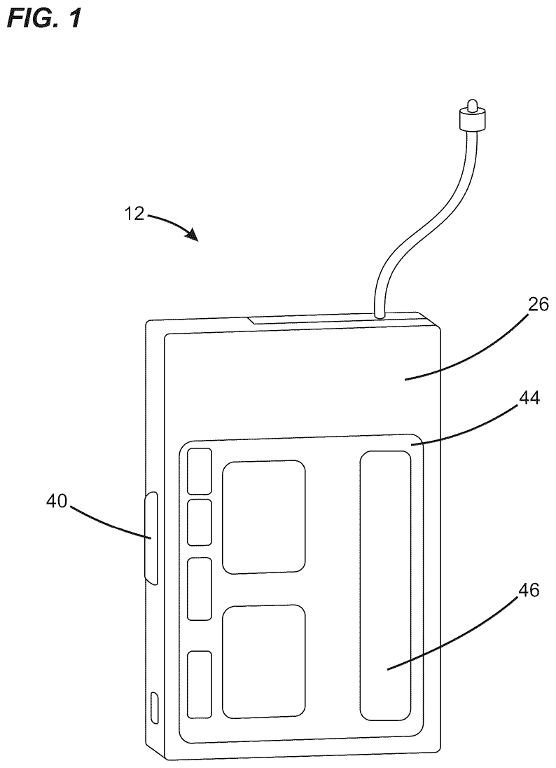

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote control device, a laptop computer, personal computer, tablet computer, a mobile communication device such as a smartphone, a wearable electronic watch or electronic health or fitness monitor, or personal digital assistant (PDA), a CGM display etc.

In an embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

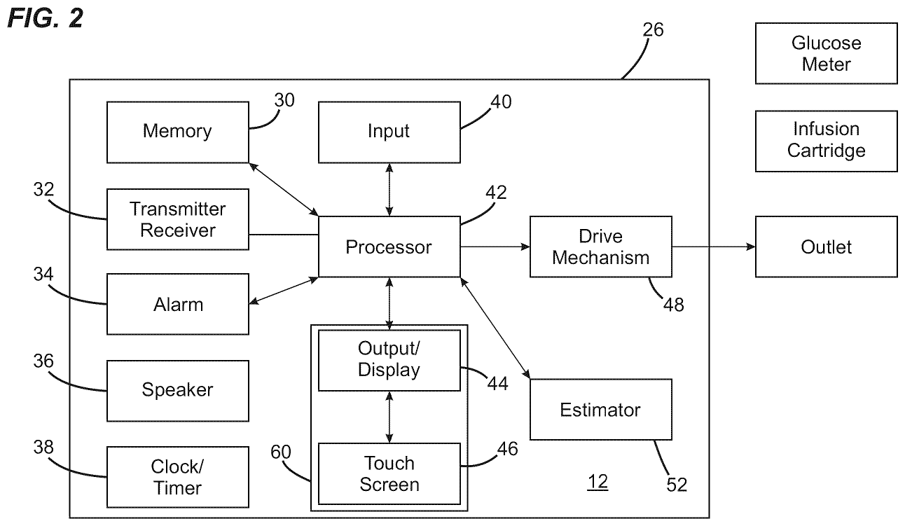

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from an input device, such as a sensor that may sense pressure, temperature or other parameters.

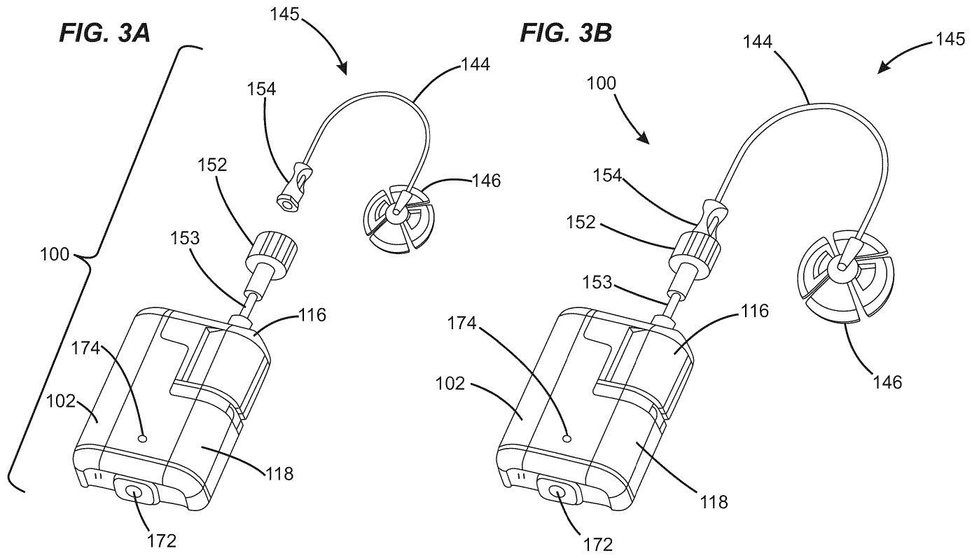

FIGS. 3A-3B depict another pump system including a pump 102 that can be used with embodiments. Drive unit 118 of pump 102 includes a drive mechanism 122 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment pump 102 does not include a display but may include one or more indicator lights 174 and/or one or more input buttons 172. Pump 102 can also incorporate any or all of the features described with respect to pump 12 in FIG. 2. Further details regarding such pumps can be found in U.S. Pat. No. 10,279,106 and U.S. Patent Publication Nos. 2016/0339172 and 2017/0049957, each of which is hereby incorporated herein by reference in its entirety.

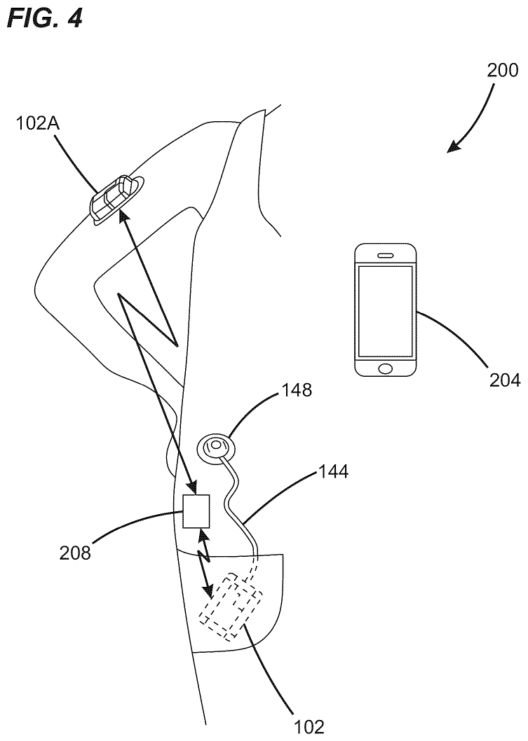

Pump 12 or 102 can interface directly or indirectly (via, e.g., a smartphone or other device) with a glucose meter, such as a blood glucose meter (BGM) or a continuous glucose monitor (CGM) or other glucose monitor. Referring to FIG. 4, an exemplary CGM system 100 according to an embodiment of the present invention is shown (other CGM systems can be used). The illustrated CGM system includes a sensor 101 affixed to a patient 104 that can be associated with the insulin infusion device in a CGM-pump system. The sensor 101 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be associated with the patient's blood glucose (BG) level. The sensor 101 includes a sensor body 108 that transmits data associated with the interstitial fluid to which the sensor probe 106 is exposed. The data may be transmitted from the sensor 101 to the glucose monitoring system receiver 100 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 101 to the monitoring system 100. Transmission of sensor data to the glucose monitoring system receiver by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. Alternatively, the system can automatically suggest a change in therapy upon receiving the CGM data such as an increased insulin basal rate or delivery of a bolus, but can require the user to accept the suggested change prior to delivery rather than automatically delivering the therapy adjustments.

If the CGM data indicates that the user has a low blood glucose level or hypoglycemia, the system can, for example, automatically reduce a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the user, e.g., ingest carbohydrates and/or take other actions and/or make other suggestions as may be appropriate to address the hypoglycemic condition, singly or in any desired combination or sequence. Such determination can be made by the infusion pump providing therapy or by a separate device that transmits therapy parameters to the infusion pump. In some embodiments, multiple medicaments can be employed in such a system as, for example, a first medicament, e.g., insulin, that lowers blood glucose levels and a second medicament, e.g., glucagon, that raises blood glucose levels.

As with other parameters related to therapy, such thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

As noted above, one important variable in such systems is the amount of unmetabolized insulin in the user's system or insulin on board (IOB). Current pump products employ various algorithms for calculating insulin on board for a user. For example, two known methods are Swan4 hr_IOB or Swan6 hr_IOB. As is known in the art, the Swan IOB equation is IOB(t)=Σₜ₌₀⁷²(InsulinDelivered(i)−UserBasalRate)*Decay[i], in which i is an index for insulin delivery every five minutes for the past six hours and Decay is a decaying function of i that is different for Swan6 hr_IOB and Swan4 hrIob. These equations are therefore complex and can occupy a significant amount of the computational capacity of portable insulin pumps. However, Applicant has developed an algorithm that provides a simplified method of estimating IOB disclosed in PCT Patent Application No. PCT/US23/82084, filed Dec. 1, 2023 and entitled Devices, Systems and Methods for Closed and Semi-Closed Loop Operation of Infusion Pumps, which is incorporated by reference herein.

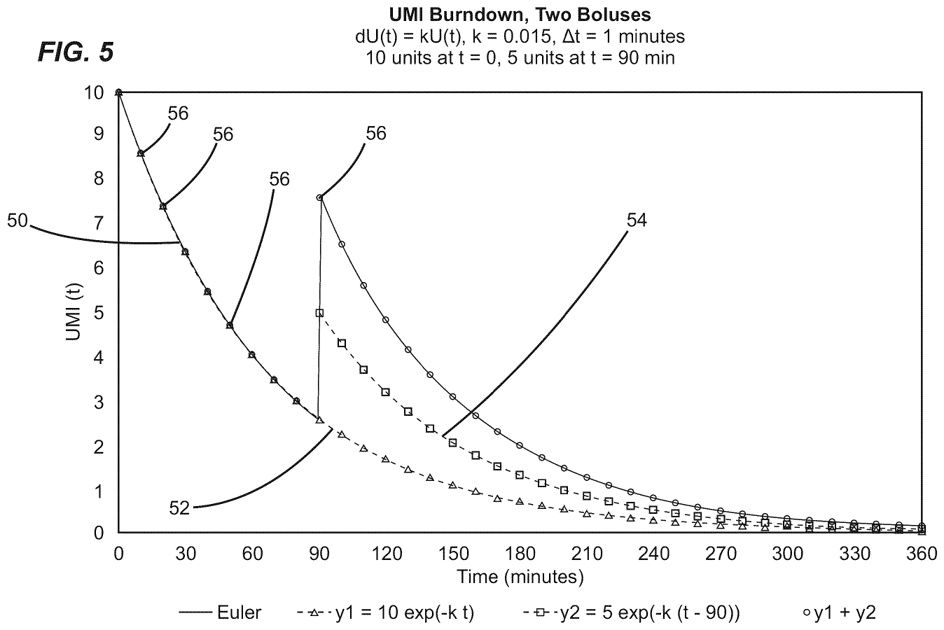

This method is based on the fact that the underlying equations for calculating IOB are linear, which means that a seemingly complex burndown of insulin can be reduced to a single addition and multiplication every five minutes. In this algorithm, the estimation for unmetabolized insulin (UMI) for a dose U₀ at time t is U(t)=U₀e^{-kt}, in which k is a burn down constant. Because this equation is linear, the burndowns from multiple doses will combine linearly. For example, the solution for a dose at t=5 and another does at t=15 can be found by summing the two burndowns. FIG. 5 depicts this principle by showing the burndown from two boluses, a ten-unit bolus delivered at t=0 and a five-unit bolus delivered at t=90. The solid line 50 depicts the sum of the boluses using Euler's method as is known in the art. The dashed line 52 with triangles depicts the burndown calculated using the above equation for the ten-unit bolus and the dashed line 54 with squares depicts the burndown calculated using the above equation for the five-unit bolus. The circular markers 56 depict the sum of the question for the ten-unit bolus and the five-unit bolus. The circular markers fall directly on the solution using Euler's method, confirming the linearity approach described herein. The nature of the exponential function enables estimation of the burndown by simple, recursive multiplications. As such, the burndown may be estimated each major timeframe by 1) adding in the new dose and 2) multiplying the same by a constant.

This simplified insulin on board estimation enables infusion pumps to expand capabilities relating to insulin on board calculations beyond those that are currently possible with the complex algorithms currently employed on these devices having limited computational capacities. For example, insulin pump users have reported a phenomenon related to “stubborn highs” in which after delivering a meal bolus if the user's glucose level does not lower as quickly as expected a user may lose patience and give one or more additional boluses including extremely high (and dangerous) boluses. In some circumstances, users may give such boluses in other forms such as in nasal form, which can cause multiple problems. First, these additional boluses will be unknown to the pump and not accounted for in the IOB tracked by the pump, which can lead to further dangerous subsequent boluses. Second, even if the pump were to be aware of such other insulin deliveries, nasal insulin, as an example, acts significantly faster than typical insulins delivered by the pump and the pump would not have the computational capacity to account for both forms of insulin. This is because a separate calculation with a separate constant would be required for the different type of insulin. Other circumstances also exist where a user may take insulin in another form than from the pump. However, if a pump employing the simplified IOB estimation described herein is provided with the information regarding the other type of bolus, this simplified calculation would enable the pump to track IOB for the different boluses simultaneously.

In embodiments, an infusion pump system can be provided with a user interface that enables a user to enter use of other insulins not delivered by the pump. For example, a user-selectable menu item can enable a user to enter both a type of insulin and an amount of insulin. In some embodiments, the user interface may include a drop down menu that lists each of the different types of insulin for which the system has a stored constant from which the user may select. The user interface may also be able to enable a user to indicate when the dose was taken so that if there has been a delay between when the user took the bolus and when the user enters the bolus into the user interface, the system can account for that time and burndown the IOB remaining from the bolus appropriately. Such a user interface can be provided on, for example, the pump itself, a smartphone or other remote control device for the pump, etc.

The pump system can then employ the simplified IOB estimation disclosed herein to separately and independently track the burndown of each bolus. At any given time, the total IOB for the user can be determined by summing the remaining IOB for the separate boluses. For example, if the user takes a bolus with the pump and a bolus of nasal insulin, when the user enters into the pump that the nasal insulin bolus was taken the pump will track that bolus independently because the burndown constant for each insulin will be different. This approach enables the system to account for different and faster or slower acting insulins than those employed by the pump for which the current algorithms are optimized. The computational complexities of current insulin delivery algorithms are such that tracking two or more insulins simultaneously in this manner is impossible to carry out on an insulin pump because of both memory and processing constraints. However, with the methods disclosed herein multiple insulin burndowns can be separately tracked with a fraction of the computational burden of current algorithms. This provides a safer system that prevents too much insulin from being delivered from the pump algorithm not being aware of insulin not delivered by the pump. A further advantage is that there is significant additional available memory and processing capacity available on pumps that employ this method that can be put to other uses.

In embodiments, a portable infusion pump system includes a pump mechanism configured to facilitate delivery of insulin to a user, a user interface and at least one processor functionally linked to the pump mechanism and the user interface. The at least one processor can be configured to cause the pump mechanism to deliver insulin doses to the user. An indication for user input can be displayed on the user interface for the portable infusion pump system to receive information pertaining to insulin delivered to the user without use of the pump mechanism. An amount of insulin on board remaining in the user can be tracked over time from both the insulin doses delivered with the pump mechanism and the insulin delivered to the user without use of the pump mechanism. A combination of the remaining insulin on board from the insulin doses delivered with the pump mechanism and the insulin delivered to the user without use of the pump mechanism can be utilized to determine a total insulin on board in making therapy determinations.

In some embodiments, the at least one processor is configured to display the indication for user input on the user interface by displaying a user-selectable menu item pertaining to insulin delivered to the user without use of the pump mechanism.

In some embodiments, the at least one processor is configured to receive user input indicating an amount of insulin delivered to the user without the use of the pump mechanism.

In some embodiments, the at least one processor is configured to receive user input indicating a type of insulin delivered to the user without the use of the pump mechanism.

In some embodiments, the at least one processor is configured to display the indication for user input on the user interface by displaying a plurality of different types of insulin for selection by the user.

In some embodiments, the plurality of different types of insulin are displayed in a drop down menu.

In some embodiments, a memory can store an insulin burndown constant for each of the different types of insulin.

In some embodiments, the at least one processor is configured to receive user input indicating a time since insulin was delivered to the user without use of the pump mechanism.

In some embodiments, the at least one processor is configured to account for the time since insulin was delivered to the user without use of the pump mechanism in tracking the amount of insulin on board remaining in the user over time for the insulin delivered to the user without use of the pump mechanism.

In some embodiments, the at least one processor is configured to separately track the amount of insulin on board remaining in the user over time from both the insulin doses delivered with the pump mechanism and the insulin delivered to the user without use of the pump mechanism.

In embodiments, a portable infusion pump system can include a pump mechanism configured to facilitate delivery of insulin to a user and at least one processor functionally linked to the pump mechanism. The at least one processor can be configured to cause the pump mechanism to deliver insulin doses to the user. Information can be received pertaining to insulin delivered to the user without use of the pump mechanism. An amount of insulin on board remaining in the user over time can be tracked from both the insulin doses delivered with the pump mechanism and the insulin delivered to the user without use of the pump mechanism. A combination of the remaining insulin on board from the insulin doses delivered with the pump mechanism and the insulin delivered to the user without use of the pump mechanism can be utilized in making therapy determinations.

In some embodiments, the at least one processor is configured to receive the information through a user-selectable menu item displayed on a user interface pertaining to insulin delivered to the user without use of the pump mechanism.

In some embodiments, the information includes an amount of insulin delivered to the user without the use of the pump mechanism.

In some embodiments, the information includes a type of insulin delivered to the user without the use of the pump mechanism.

In some embodiments, the at least one processor is configured to display a plurality of different types of insulin for selection by the user on a user interface.

In some embodiments, the plurality of different types of insulin are displayed in a drop-down menu.

In some embodiments, a memory can store an insulin burndown constant for each of the different types of insulin.

In some embodiments, the information includes a time since insulin was delivered to the user without use of the pump mechanism.

In some embodiments, the at least one processor is configured to account for the time since insulin was delivered to the user without use of the pump mechanism in tracking the amount of insulin on board remaining in the user over time for the insulin delivered to the user without use of the pump mechanism.

In some embodiments, the at least one processor is configured to separately track the amount of insulin on board remaining in the user over time from both the insulin doses delivered with the pump mechanism and the insulin delivered to the user without use of the pump mechanism.

Although primarily described in the context of tracking IOB for multiple different types of insulins having different characteristics, it should be noted that the methods described herein can also be used to track additional medicaments taken by the user that affect glucose levels, such as glucagon, pramlintide, etc. In addition, although described as a manner in which an additional insulin input can be tracked, the methods can be employed to add a negative component into the tracking of IOB. For example, if the pump has been disconnected from the user such that the pump delivered insulin that was not actually injected into the user, there is no insulin on board in the user from that non-delivered insulin. The user may be able to indicate to the pump a timeframe over which insulin was not actually received by the user and the system can use the disclosed methods to remove that component from the IOB.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing

### DETAILED DESCRIPTION

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10,541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924 and commonly owned U.S. patent application Ser. Nos. 17/368,968; 17/896,492; 18/475,916; 18/478,552; 18/678,130; 18/207,094; 18/398,543; 18/441,735; 18/474,839; 18/700,168; 18/891,482; 18/896,045; and Ser. No. 18/962,169.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A portable infusion pump system comprising:
a pump mechanism configured to facilitate delivery of insulin to a user;
a user interface; and
at least one processor functionally linked to the pump mechanism and the user interface, the at least one processor configured to:
cause the pump mechanism to deliver insulin doses to the user;
display an indication for user input on the user interface for the portable infusion pump system to receive information pertaining to insulin delivered to the user without use of the pump mechanism;
track an amount of insulin on board remaining in the user over time from both the insulin doses delivered with the pump mechanism and the insulin delivered to the user without use of the pump mechanism; and
utilize a combination of the remaining insulin on board from the insulin doses delivered with the pump mechanism and the insulin delivered to the user without use of the pump mechanism to determine a total insulin on board in making therapy determinations.

2. The portable infusion pump system of claim 1, wherein the at least one processor is configured to display the indication for user input on the user interface by displaying a user-selectable menu item pertaining to insulin delivered to the user without use of the pump mechanism.

3. The portable infusion pump system of claim 1, wherein the at least one processor is configured to receive user input indicating an amount of insulin delivered to the user without the use of the pump mechanism.

4. The portable infusion pump system of claim 1, wherein the at least one processor is configured to receive user input indicating a type of insulin delivered to the user without the use of the pump mechanism.

5. The portable infusion pump system of claim 1, wherein the at least one processor is configured to display the indication for user input on the user interface by displaying a plurality of different types of insulin for selection by the user.

6. The portable infusion pump system of claim 5, wherein the plurality of different types of insulin are displayed in a drop down menu.

7. The portable infusion pump system of claim 5, further comprising a memory storing an insulin burndown constant for each of the different types of insulin.

8. The portable infusion pump system of claim 1, wherein the at least one processor is configured to receive user input indicating a time since insulin was delivered to the user without use of the pump mechanism.

9. The portable infusion pump system of claim 8, wherein the at least one processor is configured to account for the time since insulin was delivered to the user without use of the pump mechanism in tracking the amount of insulin on board remaining in the user over time for the insulin delivered to the user without use of the pump mechanism.

10. The portable infusion pump system of claim 1, wherein the at least one processor is configured to separately track the amount of insulin on board remaining in the user over time from both the insulin doses delivered with the pump mechanism and the insulin delivered to the user without use of the pump mechanism.

11. A portable infusion pump system comprising:
a pump mechanism configured to facilitate delivery of insulin to a user; and
at least one processor functionally linked to the pump mechanism, the at least one processor configured to:
cause the pump mechanism to deliver insulin doses to the user;
receive information pertaining to insulin delivered to the user without use of the pump mechanism;
track an amount of insulin on board remaining in the user over time from both the insulin doses delivered with the pump mechanism and the insulin delivered to the user without use of the pump mechanism; and
utilize a combination of the remaining insulin on board from the insulin doses delivered with the pump mechanism and the insulin delivered to the user without use of the pump mechanism in making therapy determinations.

12. The portable infusion pump system of claim 11, wherein the at least one processor is configured to receive the information through a user-selectable menu item displayed on a user interface pertaining to insulin delivered to the user without use of the pump mechanism.

13. The portable infusion pump system of claim 11, wherein the information includes an amount of insulin delivered to the user without the use of the pump mechanism.

14. The portable infusion pump system of claim 11, wherein the information includes a type of insulin delivered to the user without the use of the pump mechanism.

15. The portable infusion pump system of claim 1, wherein the at least one processor is configured to display a plurality of different types of insulin for selection by the user on a user interface.

16. The portable infusion pump system of claim 15, wherein the plurality of different types of insulin are displayed in a drop-down menu.

17. The portable infusion pump system of claim 15, further comprising a memory storing an insulin burndown constant for each of the different types of insulin.

18. The portable infusion pump system of claim 11, wherein the information includes a time since insulin was delivered to the user without use of the pump mechanism.

19. The portable infusion pump system of claim 18, wherein the at least one processor is configured to account for the time since insulin was delivered to the user without use of the pump mechanism in tracking the amount of insulin on board remaining in the user over time for the insulin delivered to the user without use of the pump mechanism.

20. The portable infusion pump system of claim 11, wherein the at least one processor is configured to separately track the amount of insulin on board remaining in the user over time from both the insulin doses delivered with the pump mechanism and the insulin delivered to the user without use of the pump mechanism.
